(19) United States
(12) Patent Application Publication
(10) Pub. No.: US 2021/0338598 A1
(43) Pub. Date: Nov. 4, 2021

(54) DRUG-LOADED COMPOSITE NANOFIBER MEMBRANE SYSTEM, METHOD FOR PREPARING THE SAME, AND APPLICATION THEREOF

(71) Applicant: Shenzhen Guangyuan Biomaterial Co., Ltd., Shenzhen (CN)

(72) Inventors: Zhichao HAN, Shenzhen (CN); Shanshan XU, Shenzhen (CN); Jia'en WU, Shenzhen (CN); Lan WANG, Shenzhen (CN)

(21) Appl. No.: 17/378,753

(22) Filed: Jul. 18, 2021

Related U.S. Application Data
(63) Continuation-in-part of application No. PCT/CN2019/106836, filed on Sep. 20, 2019.

(30) Foreign Application Priority Data
Mar. 15, 2019 (CN) 201910198279.1

(51) Int. Cl. A61K 9/70; A61K 31/337; A61K 31/704; A61K 31/513; A61K 33/243; A61K 47/34; D01D 5/00; D04H 1/728

(52) U.S. Cl. CPC A61K 9/7007; A61K 31/337; A61K 31/704; D04H 1/728; A61K 33/243; A61K 47/34; D01D 5/0007; A61K 31/513

(57) ABSTRACT

A drug-loaded composite nanofiber membrane system, the system including a first nanofiber layer, a second nanofiber layer, and a third nanofiber layer. The first nanofiber layer includes a poly(lactic-co-glycolic acid) copolymer, poly(p-dioxanone) and a drug. The second nanofiber layer includes the poly(lactic-co-glycolic acid) copolymer, polyglycolic acid and the drug. The third nanofiber layer includes the poly(lactic-co-glycolic acid) copolymer, polyethylene glycol and the drug.

### CROSS-REFERENCE TO RELAYED APPLICATIONS

This application is a continuation-in-part of International Patent Application No. PCT/CN2019/106836 with an international filing date of Sep. 20, 2019, designating the United States, now pending, and further claims foreign priority benefits to Chinese Patent Application No. 201910198279.1 filed Mar. 15, 2019. The contents of all of the aforementioned applications, including any intervening amendments thereto, are incorporated herein by reference. Inquiries from the public to applicants or assignees concerning this document or the related applications should be directed to: Matthias Scholl PC., Attn.: Dr. Matthias Scholl Esq., 245 First Street, 18th Floor, Cambridge, Mass. 02142.

### BACKGROUND

The disclosure relates to the field of fiber membranes, and more particularly to a drug-loaded composite nanofiber membrane system, a method for preparing the same and application thereof.

There are many risk factors for tumor recurrence after resection. The center of a tumor may be completely removed by surgical resection. However, to maintain the physiological function, a tumor organ often cannot be removed completely. Therefore, surrounding calcifications of the organ are retained, which leads to recurrence. Traditional postoperative adjuvant chemotherapy eliminates residual tumors and subclinical lesions. Chemotherapy drugs are toxic and circulate in the blood of the body, which is harmful to organs and tissues.

Electrospinning technology involves the treatment of a spinnable polymer solution under a high-voltage electric field. The charged polymer droplets form a Taylor cone on a jetting head. A large electric field force can help the droplets on the jetting head overcome the surface tension to form an air stream. The jet stream is drawn, split, and cured on a receiving device to form a nanofiber membrane. This method is widely used in synthesis of a nanofiber. A drug-loaded nanofiber membrane prepared by the electrostatic spinning method can be applied to surgical dressings. The nanofibers have a higher specific surface area thereby increasing the effective action area of the drug. Three dimensional porous structure of the nanofiber membrane is conducive to cell adhesion and proliferation; good air and moisture permeability thereof is conducive to cell growth. In addition, the nanofiber membrane can prevent the sudden release of the drug, thereby improving the utilization rate of the drug.

However, the nanofiber membrane only extends the drug release time, and cannot achieve the multi-stage release of the drug.

### SUMMARY

The disclosure provides a drug-loaded composite nanofiber membrane system, the system comprising a first nanofiber layer, a second nanofiber layer, and a third nanofiber layer. The first nanofiber layer comprises a poly(lactic-co-glycolic acid) copolymer, poly(p-dioxanone) and a drug. The second nanofiber layer comprises the poly(lactic-co-glycolic acid) copolymer, polyglycolic acid and the drug. The third nanofiber layer comprises the poly(lactic-co-glycolic acid) copolymer, polyethylene glycol and the drug.

The first nanofiber layer, the second nanofiber layer, and the third nanofiber layer are stacked in an arbitrary order as needed.

The three nanofiber layers of the composite nanofiber membrane system comprise the poly(lactic-co-glycolic acid) copolymer as a main component. The poly(lactic-co-glycolic acid) copolymer is a hydrophobic functional polymer with good biocompatibility and biodegradability, can be implanted in the body and exhibit good film-forming property. However, the poly(lactic-co-glycolic acid) copolymer has poor hydrophilicity, high crystallinity, and low water absorption, and thus the nanofiber layers are degraded very slowly. Different hydrophilic polymers are added as second components in each layer. Based on the interaction between the polymers and the interaction between the polymers and the drugs, a drug slow-release system lasting for 0 day-2.5 months (0 day means the drug is released in a few seconds) is established, thereby achieving multi-gradient, multi-stage and long-acting drug release.

The first nanofiber layer has the fastest drug release rate, which is adjusted through the mass percentage of poly(p-dioxanone) in the fiber layer. The second nanofiber layer has a relatively slow drug release rate, which is adjusted through the mass percentage of polyglycolic acid in the fiber layer. The third nanofiber layer has the slowest drug release rate, which is adjusted through a molar ratio of lactic acid and hydroxyacetic acid in the poly(lactic-co-glycolic acid) copolymer. The multi-gradient and multi-stage long-acting drug release lasts for as long as two and a half months.

In a class of this embodiment, the poly(lactic-co-glycolic acid) copolymer has a viscosity average molecular weight of 40,000-250,000 Da, such as 40,000 Da, 50,000 Da, 60,000 Da, 80,000 Da, 100,000 Da, 120,000 Da, 140,000 Da, 160,000 Da, 200,000 Da or 250,000 Da, preferably 40,000 to 120,000 Da.

The molecular weight of the poly(lactic-co-glycolic acid) copolymer reflects the number of entanglement of a polymer molecular chain in a solution. The viscosity of the polymer solution increases with the increase of its molecular weight. Too low molecular weight forms droplets rather than continuous fibers. Preferably, the poly(p-dioxanone) has an intrinsic viscosity of 1-10 dL/g, for example, 1 dL/g, 2 dL/g, 3 dL/g, 4 dL/g, 5 dL/g, 6 dL/g, 7 dL/g, 8 dL/g, 9 dL/g, or 10 dL/g, preferably 1-5 dL/g.

The intrinsic viscosity of poly-dioxanone has an effect on the drug release rate of the first nanofiber layer. The greater the intrinsic viscosity of poly-dioxanone, the slower the drug release rate of the first nanofiber layer. However, the effect of the intrinsic viscosity on the drug release rate is relatively weak compared with the mass percentage of poly (p-dioxanone).

In a class of this embodiment, the polyglycolic acid has an intrinsic viscosity of 0.5-10 dL/g, such as 0.5 dL/g, 0.8 dL/g, 1 dL/g, 2 dL/g, 4 dL/g, 5 dL/g, 8 dL/g, or 10 dL/g, preferably 0.5-5 dL/g.

The intrinsic viscosity of polyglycolic acid has an effect on the drug release rate of the first nanofiber layer. The greater the intrinsic viscosity of polyglycolic acid, the slower the drug release rate of the second nanofiber layer. However, the effect of the intrinsic viscosity on the drug release rate is relatively weak compared with the mass percentage of polyglycolic acid.

In a class of this embodiment, polyethylene glycol has a viscosity average molecular weight of 1000-20000 Da, such as 1000 Da, 2000 Da, 4000 Da, 5000 Da, 6000 Da, 8000 Da, 10000 Da, 12000 Da, 14000 Da, 16000 Da, 18000 Da, or 20000 Da, preferably 2000-10000 Da.

The molecular weight of polyethylene glycol influences the swelling and diffusion channels on the fiber surface. Polyethylene glycol with too small molecular weight (for example, smaller than 1000 Da) has no regulatory effect and is similar to the drug. Polyethylene glycol with too large molecular weight (for example, larger than 20000 Da) cannot be metabolized in the body, with poor compatibility with PLGA, resulting in phase separation and a burst release of the drug.

In a class of this embodiment, a mass ratio of the poly(lactic-co-glycolic acid) copolymer to poly(p-dioxanone) in the first nanofiber layer is between 70:30 and 97:3, such as 70:30, 75:25, 78:22, 80:20, 82:18, 85:15, 88:12, 90:10, 93:7, 95:5, or 97:3.

Controlling the mass ratio of the poly(lactic-co-glycolic acid) copolymer to poly(p-dioxanone) within the range of between 70:30 and 97:3 can control the release period of the drug in the first nanofiber layer within 7 days. The greater the mass ratio, the longer the drug release period.

In a class of this embodiment, the molar ratio of lactic acid unit to hydroxyacetic acid unit in the poly(lactic-co-glycolic acid) copolymer in the first nanofiber layer is greater than or equal to 1:1, for example, 1:1, 2:1, 3:1, 4:1, 5:1, 6:1, 7:1, 8:1, 9:1, or 9.9:0.1.

In a class of this embodiment, the drug in the first nanofiber layer is taxol, doxorubicin, cis-platinum, carboplatin, 5-fluorouracil, or a combination thereof, for example, a combination of taxol and doxorubicin, a combination of cis-platinum and carboplatin, and a combination of carboplatin and 5-fluorouracil.

The first nanofiber layer can be subdivided into two or more fiber sublayers. A mass ratio of the poly(lactic-co-glycolic acid) copolymer to poly(p-dioxanone) in each sublayer is between 70:30 and 97:3, which can be an arbitrary value according to actual needs. The molar ratio of the lactic acid to the hydroxyacetic acid structural unit in the poly(lactic-co-glycolic acid) copolymer in each sublayer is greater than or equal to 1:1, of which value can be arbitrarily selected according to actual needs.

In a class of this embodiment, the mass ratio of the poly(lactic-co-glycolic acid) copolymer to polyglycolic acid in the second nanofiber layer is between 60:40 and 99:1, such as 60:40, 65:35, 70:30, 75:25, 80:20, 85:15, or 90:10, 99:1.

Controlling the mass ratio of the poly(lactic-co-glycolic acid) copolymer to polyglycolic acid within between 6:4 and 9:1 can control the drug release cycle in the second nanofiber layer within 7 days to 1 month. The greater the mass ratio, the longer the drug release period.

In a class of this embodiment, the molar ratio of lactic acid unit to hydroxyacetic acid unit in the poly(lactic-co-glycolic acid) copolymer in the second nanofiber layer is greater than or equal to 1:1, such as 1:1, 2:1, 3:1, 3.5:1, 4:1, 5:1, 5.5:1, 6:1, 7:1, 8:1, or 9:1.

In a class of this embodiment, the drug in the second nanofiber layer is taxol, doxorubicin, cis-platinum, carboplatin, 5-fluorouracil, or a combination thereof, for example, a combination of taxol and doxorubicin, a combination of cis-platinum and carboplatin, and a combination of carboplatin and 5-fluorouracil.

The second nanofiber layer can be subdivided into two or more fiber sublayers. A mass ratio of the poly(lactic-co-glycolic acid) copolymer to polyglycolic acid in each sublayer is between 6:4 and 9:1, which can be an arbitrary value according to actual needs. The molar ratio of the lactic acid to the hydroxyacetic acid structural unit in the poly(lactic-co-glycolic acid) copolymer in each sublayer is greater than or equal to 1:1, of which value can be arbitrarily selected according to actual needs.

In a class of this embodiment, the mass ratio of the poly(lactic-co-glycolic acid) copolymer to polyethylene glycol in the third nanofiber layer is between 70:30 and 97:3, such as 70:30, 75:25, 78:22, 80:20, 82:18, 85:15, 88:12, 90:10, 93:7, 95:5, or 97:3.

In a class of this embodiment, the molar ratio of the lactic acid to the hydroxyacetic acid in the poly(lactic-co-glycolic acid) copolymer in the third nanofiber layer is greater than or equal to 1:1, such as 1:1, 2:1, 3:1, 3.5:1, 4:1, 5:1, 5.5:1, 6:1, 7:1, 8:1, or 9:1.

Controlling the molar ratio of lactic acid unit to hydroxyacetic acid unit in the poly(lactic-co-glycolic acid) copolymer within (1-9):1 can control the drug release cycle in the third nanofiber layer within a range from 1 month to 2.5 months. The greater the molar ratio, the longer the drug release period.

In a class of this embodiment, the drug in the third nanofiber layer is taxol, doxorubicin, cis-platinum, carboplatin, 5-fluorouracil, or a combination thereof, for example, a combination of taxol and doxorubicin, a combination of cis-platinum and carboplatin, and a combination of carboplatin and 5-fluorouracil.

The third nanofiber layer can be subdivided into two or more fiber sublayers. A mass ratio of the poly(lactic-co-glycolic acid) copolymer to polyethylene glycol in each sublayer is 70:30-97:1, which can be an arbitrary value according to actual needs. The molar ratio of the lactic acid to the hydroxyacetic acid structural unit in the poly(lactic-co-glycolic acid) copolymer in each sublayer is to (1-9):1, of which value can be arbitrarily selected according to actual needs.

In a class of this embodiment, in the first nanofiber layer, the mass ratio of the drug to the polymers is between 1:4 and 1:10.

In a class of this embodiment, in the second nanofiber layer, the mass ratio of the drug to the polymers is between 1:4 and 1:10.

In a class of this embodiment, in the third nanofiber layer, the mass ratio of the drug to the polymers is between 1:4 and 1:10.

In the first nanofiber layer, the second nanofiber layer, and the third nanofiber layer, a mass ratio of each layer of drug to each layer of polymer is between 1:4 and 1:10, such as, 1:4, 1:5, 1:5.5, 1:6, 1:7, 1:7.5, 1:8, 1:8.5, 1:9, or 1:10.

The mass ratio of each layer of drug to each layer of polymer is controlled within the range of between 1:4 and 1:10. The higher ratio causes sudden drug release, and a large amount of release causes toxicity due to a too high local drug concentration. The lower ratio cannot reach an effective onset concentration.

The poly(lactic-co-glycolic acid) copolymer in the first nanofiber layer, the second nanofiber layer or the third nanofiber layer can be a mixture of two or more poly(lactic-co-glycolic acid) copolymers with different LA/GA molar ratios.

In another aspect, the disclosure provides a method for preparing a drug-loaded composite nanofiber membrane system as described above, the method comprising:
(1) respectively dissolving and mixing polymers and the drug according to raw materials of three nanofiber layers to obtain three mixed solutions; and
(2) sequentially introducing the three mixed solutions in 1) for electrostatic spinning to obtain the drug-loaded composite nanofiber membrane system.

The drug-loaded composite nanofiber membrane system described in the disclosure is made by degradable polymers through electrostatic spinning, is stable in nature and has high porosity, similar to an extracellular matrix, and is applied to a postoperative stump without the need for secondary surgery, and can be degraded in the body.

In a class of this embodiment, (1) is performed as follows: dissolving the drug for each nanofiber layer in a solvent, and adding polymers for each nanofiber layer in a mixture of the drug and solvent, stirring and mixing, thereby obtaining the three mixed solutions.

In a class of this embodiment, the solvent is N,N-dimethylformamide, acetone, hexafluoroisopropanol, or a combination thereof, for example, a combination of N,N-dimethylformamide and acetone, a combination of acetone and hexafluoroisopropanol, and a combination of N,N-dimethylformamide and hexafluoroisopropanol, etc.

In a class of this embodiment, an inner diameter of a spinneret is 0.4 mm during electrostatic spinning.

In a class of this embodiment, a voltage during electrostatic spinning is 10-25 kV, such as 10 kV, 12 kV, 13 kV, 14 kV, 15 kV, 16 kV, 18 kV, 20 kV, 22 kV, 24 kV, or 25 kV, preferably 20-25 kV.

In a class of this embodiment, a spinning distance during the electrostatic spinning is 5-15 cm, such as 5 cm, 6 cm, 7 cm, 8 cm, 9 cm, 10 cm, 12 cm, 14 cm, or 15 cm, preferably 8-15 cm.

In a class of this embodiment, a temperature for electrostatic spinning is 20-30° C., such as 20° C., 21° C., 22° C., 23° C., 24° C., 25° C., 26° C., 27° C., 28° C., 29° C., or 30° C.

In a class of this embodiment, an advancing speed of each mixed solution during the electrostatic spinning is 4-10 mL/L, for example, 4 mL/L, 5 mL/L, 6 mL/L, 7 mL/L, 8 mL/L, 9 mL/L, or 10 mL/L, preferably 6-10 mL/L.

In a class of this embodiment, a receiving device during the electrostatic spinning is a metal drum with a diameter of 5 cm, and a rotation speed is 600-900 rpm, such as 600 rpm, 650 rpm, 700 rpm, 750 rpm, 800 rpm, 850 rpm, or 900 rpm, preferably 800 rpm.

In a class of this embodiment, in (2), the drug-loaded composite nanofiber membrane system is post-processed as follows: the drug-loaded composite nanofiber membrane system is vacuum-dried at 20-30° C. (for example, 20° C., 21° C., 22° C., 23° C., 24° C., 25° C., 26° C., 27° C., 28° C., 29° C. or 30° C., etc.) for 24-72 h (24 h, 30 h, 35 h, 50 h, 60 h or 72 h, etc.).

Specifically, the method comprises the following steps:
(1) dissolving the drug for each nanofiber layer in a solvent, and adding polymers for each nanofiber layer in a mixture of the drug and solvent, stirring and mixing, thereby obtaining the three mixed solutions;
(2) respectively loading the three mixed solutions in (1) into a 22G flat-head dispensing syringe for electrostatic spinning at 20-30° C., where the spinneret has an inner diameter of 0.4 mm; the advancing speed of each mixed solution is 4-10 mL/L, the spinning voltage is 10-25 kV, the spinning distance is 5-15 cm, the receiving device is the metal drum with the diameter of 5 cm; the rotation speed of the metal drum is 600-900 rpm; and
(3) vacuum-drying the drug-loaded composite nanofiber membrane system in (2) at 20-30° C. for 24-72 h.

In another aspect, the disclosure provides an application of a drug-loaded composite nanofiber membrane system in the preparation of an anti-tumor drug.

The following advantages are associated with the drug-loaded composite nanofiber membrane system of the disclosure:

1. The drug-loaded composite nanofiber membrane of the disclosure adds different hydrophilic polymers as a second component to the main component of each layer, establishes a complete drug release system of 0 days to 2.5 months, thereby achieving multi-gradient and multi-stage long-acting drug release and efficacy.

2. The drug-loaded composite nanofiber membrane system described in the disclosure is made by electrostatic spinning using a degradable polymer, is stable in nature and has high porosity, similar to an extracellular matrix, and is applied to a postoperative stump without the need for secondary surgery, and can be degraded in the body.

### BRIEF DESCRIPTION OF THE DIAGRAMS

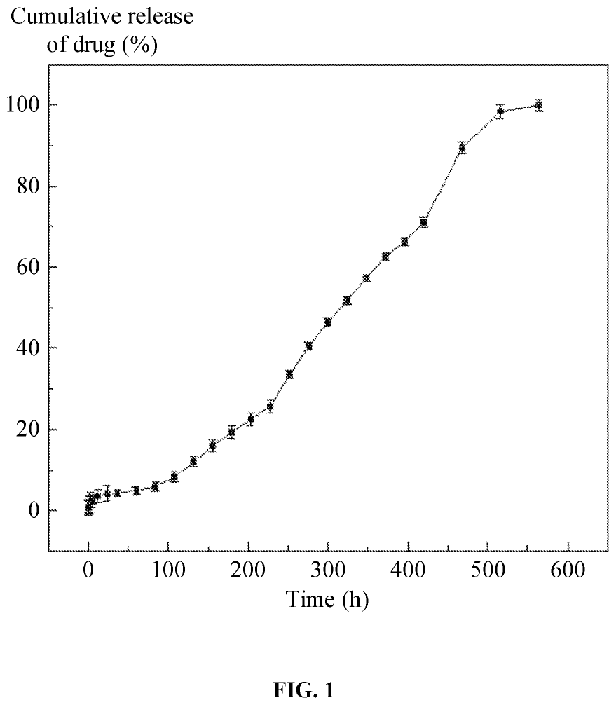

FIG. 1 is a first drug release curve of a drug-loaded composite nanofiber membrane system prepared in Example 1;

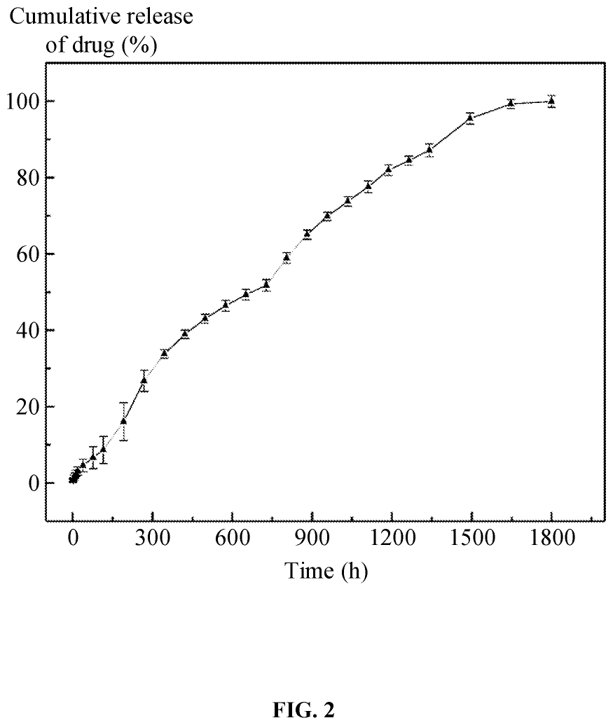

FIG. 2 is a second drug release curve of a drug-loaded composite nanofiber membrane system prepared in Example 1;

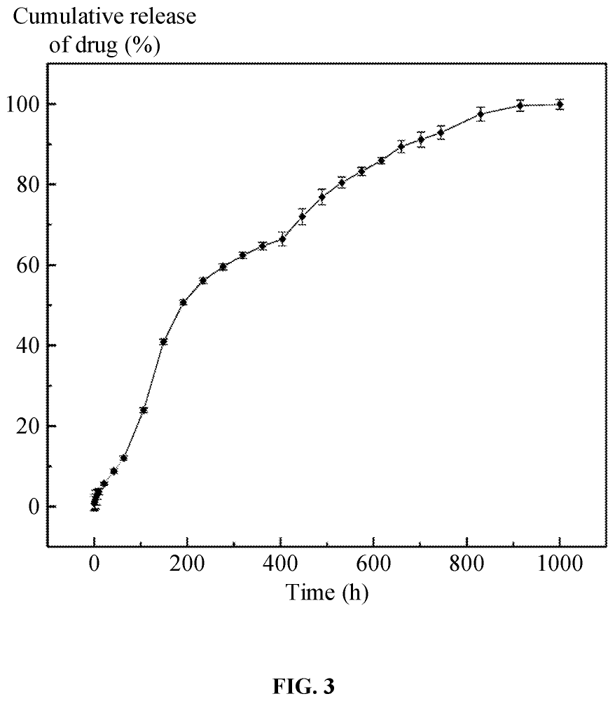

FIG. 3 is a third drug release curve of a drug-loaded composite nanofiber membrane system prepared in Example 1; and

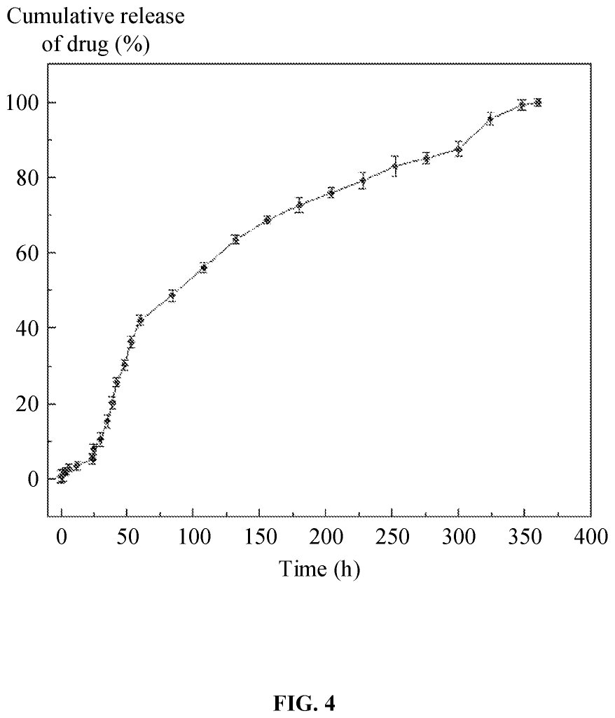

FIG. 4 is a fourth drug release curve of a drug-loaded composite nanofiber membrane system prepared in Example 1.

### DESCRIPTION OF THE INVENTION

To further illustrate, embodiments detailing a drug release curve of a drug-loaded composite nanofiber membrane system are described below. It should be noted that the following embodiments are intended to describe and not to limit the disclosure.

### Example 1

This example provided a drug-loaded composite nanofiber membrane system, comprising a first nanofiber layer, a second nanofiber layer, and a third nanofiber layer.

The first nanofiber layer comprised poly(lactic-co-glycolic acid) (PLGA) copolymer (molecular weight of 60000), poly(p-dioxanone) (PDO) (intrinsic viscosity of 1.2-2.4 dL/g) and taxol. A mass ratio of PLGA to PDO was 9:1. Taxol accounted for 15% of the total mass of PLGA and PDO, and a molar ratio of lactic acid (LA) to GA in PLGA was 1:1.

The second nanofiber layer comprised poly(lactic-co-glycolic acid) (PLGA) copolymer (molecular weight of 80,000), polyglycolic acid (PGA) (intrinsic viscosity of 0.5-1.8 dL/g) and taxol. A mass ratio of PLGA to PGA was 93:7. Taxol accounted for 10% of the total mass of PLGA and PGA, and the molar ratio of LA to GA in PLGA was 3:1.

The third nanofiber layer comprised poly(lactic-co-glycolic acid) (PLGA) copolymer (molecular weight of 80,000), polyethylene glycol (PEG) (molecular weight of 2000) and taxol. A mass ratio of PLGA to PEG was 95:5. Taxol accounted for 20% of the total mass of PLGA and PEG, and the molar ratio of LA to GA in PLGA was 3:1.

The method for preparing the drug-loaded composite nanofiber membrane system was as follows:

(1) dissolving taxol for the first, second, and third nanofiber layers in hexafluoroisopropanol, acetone, and N,N-dimethylformamide, respectively, adding the polymers for each nanofiber to the three drug solutions, stirring and mixing the solutions to obtain three mixed solutions;

(2) loading the three mixed solutions in (1) into a 22G flat-head dispensing syringe for electrostatic spinning at 25° C., where the spinneret had an inner diameter of 0.4 mm; the advancing speed of each mixed solution was 4 mL/L, the spinning voltage was 25 kV, the spinning distance was 15 cm, the receiving device was a metal drum with the diameter of 5 cm; the rotation speed was 600 rpm, to yield the drug-loaded composite nanofiber membrane system; and

(3) vacuum-drying the drug-loaded composite nanofiber membrane system in (2) at 25° C. for 24 h.

The drug-loaded composite nanofiber membrane system was tested for drug release, and the drug release curve was drawn as follows: the dried drug-loaded composite nanofiber membrane system was cut into 10 mg samples, and the samples were put into a centrifuge tube with 10 mL of a fresh phosphate buffered saline (PBS) solution. Then the samples were put in an air bath constant temperature shaker, with the temperature of 37° C., and the speed of the shaker of 100 rpm. At designated time intervals, 1 mL of release solution was taken out, and an equal amount of the fresh PBS solution was added. Then, a standard curve of the drug′ concentration was measured with an ultraviolet-visible spectrophotometer, and the amount of drug released by the drug-loaded composite nanofiber membrane system was determined according to the standard curve. All experimental groups were in five copies, and the measured drug release amount was expressed as mean±standard deviation. The experimental results were shown in FIG. 1. The drug release system presented a typical three-stage release feature, with a release cycle of nearly 600 h. In the initial stage, the drug release was sustained but slow, and began to accelerate in an intermediate stage. At 420 h, the drug release rate was greatly accelerated until the drug was completely released.

### Example 2

This example provided a drug-loaded composite nanofiber membrane system, comprising a first nanofiber layer, a second nanofiber layer, and a third nanofiber layer.

The first nanofiber layer comprised poly(lactic-co-glycolic acid) (PLGA) copolymer (molecular weight of 120000), poly(p-dioxanone) (PDO) (intrinsic viscosity of 2.4-4.8 dL/g) and doxorubicin. A mass ratio of PLGA to PDO was 8:1. Doxorubicin accounted for 15% of the total mass of PLGA and PDO, and a molar ratio of LA to GA in PLGA was 1:1.

The second nanofiber layer comprised poly(lactic-co-glycolic acid) (PLGA) copolymer (molecular weight of 40000), polyglycolic acid (PGA) (intrinsic viscosity of 2.5-4.0 dL/g) and doxorubicin. A mass ratio of PLGA to PGA was 6:4. Doxorubicin accounted for 10% of the total mass of PLGA and PGA, and the molar ratio of LA to GA in PLGA was 3:1.

The third nanofiber layer comprised poly(lactic-co-glycolic acid) (PLGA) copolymer (molecular weight of 150,000), polyethylene glycol (PEG) (molecular weight of 5000) and doxorubicin. The mass ratio of PLGA to PEG was 95:5. Doxorubicin accounted for 25% of the total mass of PLGA and PEG, and contained three poly(lactic-co-glycolic acid) copolymers with different LA/GA molar ratios. The molar ratio of LA to GA and a relative mass fraction of the poly(lactic-co-glycolic acid) copolymers to the nanofiber layer were 85:15 (50%), 75:2 (25%), and 65:35 (25%), respectively.

The method for preparing the drug-loaded composite nanofiber membrane system was as follows:

(1) dissolving adriamycin in the first, second, and third nanofiber layers in hexafluoroisopropanol, acetone, and N,N-dimethylformamide, respectively, adding the polymers for each nanofiber to the three drug solutions, stirring and mixing the solutions to obtain three mixed solutions;

(2) loading the three mixed solutions in (1) into a 22G flat-head dispensing syringe for electrostatic spinning at 25° C., where the spinneret had an inner diameter of 0.4 mm; the advancing speed of each mixed solution was 6 mL/L, the spinning voltage was 20 kV, the spinning distance was 10 cm, the receiving device was a metal drum with the diameter of 5 cm; the rotation speed was 700 rpm, to yield the drug-loaded composite nanofiber membrane system; and

(3) vacuum-drying the drug-loaded composite nanofiber membrane system in (2) at 25° C. for 48 h.

The drug-loaded composite nanofiber membrane system was tested for drug release, and the drug release curve was drawn using the same method as in Example 1. The experimental results were shown in FIG. 2. The drug release system presented a typical three-stage release feature, with a release cycle of nearly 1800 h.

### Example 3

This example provided a drug-loaded composite nanofiber membrane system, comprising a first nanofiber layer, a second nanofiber layer, and a third nanofiber layer.

The first nanofiber layer comprised poly(lactic-co-glycolic acid) (PLGA) copolymer (molecular weight of 40000), poly(p-dioxanone) (PDO) (intrinsic viscosity of 2.4-4.8 dL/g) and 5-fluorouracil. A mass ratio of PLGA to PDO was 7:1. Fluorouracil accounted for 15% of the total mass of PLGA and PDO, and a molar ratio of LA to GA in PLGA was 1:1.

The second nanofiber layer comprised poly(lactic-co-glycolic acid) (PLGA) copolymer (molecular weight of 200000), polyglycolic acid (PGA) (intrinsic viscosity of 8.0-9.0 dL/g) and 5-fluorouracil. A mass ratio of PLGA to PGA was 7:3. 5-fluorouracil accounted for 10% of the total mass of PLGA and PGA, and the molar ratio of LA to GA in PLGA was 3:1.

The third nanofiber layer comprised poly(lactic-co-glycolic acid) (PLGA) copolymer (molecular weight of 150000), polyethylene glycol (PEG) (molecular weight of 10000) and 5-fluorouracil. A mass ratio of PLGA to PEG was 95:5. 5-fluorouracil accounted for 25% of the total mass of PLGA and PEG, and the molar ratio of LA to GA in PLGA was 5:1.

The method for preparing the drug-loaded composite nanofiber membrane system was as follows:

(1) dissolving 5-fluorouracil in the first, second, and third nanofiber layers in hexafluoroisopropanol, acetone, and N,N-dimethylformamide, respectively, adding the polymers for each nanofiber to the three drug solutions, stirring and mixing the solutions to obtain three mixed solutions;

(2) loading the three mixed solutions in (1) into a 22G flat-head dispensing syringe for electrostatic spinning at 25° C., where the spinneret had an inner diameter of 0.4 mm; the advancing speed of each mixed solution was 10 mL/L, the spinning voltage was 10 kV, the spinning distance was 5 cm, the receiving device was a metal drum with the diameter of 5 cm; the rotation speed was 900 rpm, to yield the drug-loaded composite nanofiber membrane system; and

(3) vacuum-drying the drug-loaded composite nanofiber membrane system in (2) at 25° C. for 72 h.

The drug-loaded composite nanofiber membrane system was tested for drug release, and the drug release curve was drawn using the same method as in Example 1. The experimental results were shown in FIG. 3. The drug release system presented a typical three-stage release feature, with a release cycle of nearly 1000 h. The drug release was fast in the initial stage, and began to slow down at 150 h, but a large amount of drug was still released. The drug was completely released in the third stage.

### Example 4

This example provided a drug-loaded composite nanofiber membrane system, comprising a first nanofiber layer, a second nanofiber layer, and a third nanofiber layer.

The first nanofiber layer comprised poly(lactic-co-glycolic acid) (PLGA) copolymer (molecular weight of 40000), poly(p-dioxanone) (PDO) (intrinsic viscosity of 2.4-4.8 dL/g) and cis-platinum. A mass ratio of PLGA to PDO was 7:3. Cis-platinum accounted for 15% of the total mass of PLGA and PDO, and a molar ratio of LA to GA in PLGA was 1:1.

The second nanofiber layer comprised poly(lactic-co-glycolic acid) (PLGA) copolymer (molecular weight of 200000), polyglycolic acid (PGA) (intrinsic viscosity of 8.0-9.0 dL/g) and cis-platinum. A mass ratio of PLGA to PGA was 6:4. Cis-platinum accounted for 10% of the total mass of PLGA and PGA, and the molar ratio of LA to GA in PLGA was 2:1.

The third nanofiber layer comprised poly(lactic-co-glycolic acid) (PLGA) copolymer (molecular weight of 150000), polyethylene glycol (PEG) (molecular weight of 10000) and cis-platinum. A mass ratio of PLGA to PEG was 7:3. Cis-platinum accounted for 25% of the total mass of PLGA and PEG, and the molar ratio of LA to GA in PLGA was 4:1.

The method for preparing the drug-loaded composite nanofiber membrane system was as follows:

(1) dissolving cisplatin in the first, second, and third nanofiber layers in hexafluoroisopropanol, acetone, and N,N-dimethylformamide, respectively, adding the polymers for each nanofiber to the three drug solutions, stirring and mixing the solutions to obtain three mixed solutions;

(2) loading the three mixed solutions in (1) into a 22G flat-head dispensing syringe for electrostatic spinning at 25° C., where the spinneret had an inner diameter of 0.4 mm; the advancing speed of each mixed solution was 10 mL/L, the spinning voltage was 25 kV, the spinning distance was 15 cm, the receiving device was a metal drum with the diameter of 5 cm; the rotation speed was 900 rpm, to yield the drug-loaded composite nanofiber membrane system; and

(3) vacuum-drying the drug-loaded composite nanofiber membrane system in (2) at 25° C. for 72 h.

The drug-loaded composite nanofiber membrane system was tested for drug release, and the drug release curve was drawn using the same method as in Example 1. The experimental results were shown in FIG. 1. The drug release system presented a typical three-stage release feature, with a release cycle of nearly 360 h. The drug release was fast in the initial stage, and began to slow down at 60 h, but a large amount of drug was still released. The drug was completely released in the third stage.

It will be obvious to those skilled in the art that changes and modifications may be made, and therefore, the aim in the appended claims is to cover all such changes and modifications.

What is claimed is:

1. A drug-loaded composite nanofiber membrane system, the system comprising:
a first nanofiber layer, the first nanofiber layer comprising a poly(lactic-co-glycolic acid) copolymer, poly(p-dioxanone), and a drug;
a second nanofiber layer, the second nanofiber layer comprising the poly(lactic-co-glycolic acid) copolymer, polyglycolic acid, and the drug; and
a third nanofiber layer, the third nanofiber layer comprising the poly(lactic-co-glycolic acid) copolymer, polyethylene glycol, and the drug.

2. The system of claim 1, wherein the poly(lactic-co-glycolic acid) copolymer has a viscosity average molecular weight of 40,000-250,000 Da; the poly(p-dioxanone) has an intrinsic viscosity of 1-10 dL/g; the polyglycolic acid has an intrinsic viscosity of 0.5-10 dL/g; and polyethylene glycol has a viscosity average molecular weight of 1000-20000 Da.

3. The system of claim 1, wherein the poly(lactic-co-glycolic acid) copolymer has a viscosity average molecular weight of 40,000-120,000 Da; the poly(p-dioxanone) has an intrinsic viscosity of 1-5 dL/g; the polyglycolic acid has an intrinsic viscosity of 0.5-5 dL/g; and polyethylene glycol has a viscosity average molecular weight of 2000-10000 Da.

4. The system of claim 1, wherein a mass ratio of the poly(lactic-co-glycolic acid) copolymer to poly(p-dioxanone) in the first nanofiber layer is between 70:30 and 97:3; and a molar ratio of lactic acid unit to hydroxyacetic acid unit in the poly(lactic-co-glycolic acid) copolymer in the first nanofiber layer is greater than or equal to 1:1.

5. The system of claim 2, wherein a mass ratio of the poly(lactic-co-glycolic acid) copolymer to poly(p-dioxanone) in the first nanofiber layer is between 70:30 and 97:3; and a molar ratio of lactic acid unit to hydroxyacetic acid unit in the poly(lactic-co-glycolic acid) copolymer in the first nanofiber layer is greater than or equal to 1:1.

6. The system of claim 3, wherein a mass ratio of the poly(lactic-co-glycolic acid) copolymer to poly(p-dioxanone) in the first nanofiber layer is between 70:30 and 97:3; and a molar ratio of lactic acid unit to hydroxyacetic acid unit in the poly(lactic-co-glycolic acid) copolymer in the first nanofiber layer is greater than or equal to 1:1.

7. The system of claim 1, wherein the drug in the first nanofiber layer is taxol, doxorubicin, cis-platinum, carboplatin, 5-fluorouracil, or a combination thereof.

8. The system of claim 1, wherein a mass ratio of the poly(lactic-co-glycolic acid) copolymer to polyglycolic acid in the second nanofiber layer is between 60:40 and 99:1; and a molar ratio of lactic acid unit to hydroxyacetic acid unit in the poly(lactic-co-glycolic acid) copolymer in the second nanofiber layer is greater than or equal to 1:1.

9. The system of claim 7, wherein a mass ratio of the poly(lactic-co-glycolic acid) copolymer to polyglycolic acid in the second nanofiber layer is between 60:40 and 99:1; and a molar ratio of lactic acid unit to hydroxyacetic acid unit in the poly(lactic-co-glycolic acid) copolymer in the second nanofiber layer is greater than or equal to 1:1.

10. The system of claim 1, wherein the drug in the second nanofiber layer is taxol, doxorubicin, cis-platinum, carboplatin, 5-fluorouracil, or a combination thereof.

11. The system of claim 1, wherein a mass ratio of the poly(lactic-co-glycolic acid) copolymer to polyethylene glycol in the third nanofiber layer is between 70:30 and 97:3; and a molar ratio of the lactic acid to the hydroxyacetic acid in the poly(lactic-co-glycolic acid) copolymer in the third nanofiber layer is greater than or equal to 1:1.

12. The system of claim 10, wherein a mass ratio of the poly(lactic-co-glycolic acid) copolymer to polyethylene glycol in the third nanofiber layer is between 70:30 and 97:3; and a molar ratio of the lactic acid to the hydroxyacetic acid in the poly(lactic-co-glycolic acid) copolymer in the third nanofiber layer is greater than or equal to 1:1.

13. The system of claim 1, wherein the drug in the third nanofiber layer is taxol, doxorubicin, cis-platinum, carboplatin, 5-fluorouracil, or a combination thereof.

14. The system of claim 1, wherein in the first nanofiber layer, a mass ratio of the drug to polymers is between 1:4 and 1:10; in the second nanofiber layer, a mass ratio of the drug to polymers is between 1:4 and 1:10; and in the third nanofiber layer, a mass ratio of the drug to polymers is between 1:4 and 1:10.

15. A method for preparing the drug-loaded composite nanofiber membrane system of claim 1, the method comprising:
1) respectively dissolving and mixing polymers and the drug according to raw materials of three nanofiber layers to obtain three mixed solutions; and
2) sequentially introducing the three mixed solutions in 1) for electrostatic spinning to obtain the drug-loaded composite nanofiber membrane system.

16. The method of claim 15, wherein 1) is performed as follows: dissolving the drug for each nanofiber layer in a solvent, and adding polymers for each nanofiber layer in a mixture of the drug and solvent, stirring and mixing, thereby obtaining the three mixed solutions;
the solvent is N,N-dimethylformamide, acetone, hexafluoroisopropanol, or a combination thereof;
an inner diameter of a spinneret is 0.4 mm during electrostatic spinning;
a voltage during electrostatic spinning is 10-25 kV;
a spinning distance during the electrostatic spinning is 5-15 cm;
a temperature for electrostatic spinning is 20-30° C.;
an advancing speed of each mixed solution during the electrostatic spinning is 4-10 mL/L;
a receiving device during the electrostatic spinning is a metal drum with a diameter of 5 cm, and a rotation speed is 600-900 rpm; and
after 2), the drug-loaded composite nanofiber membrane system is vacuum-dried at 20-30° C. for 24-72 h.

17. The method of claim 16, wherein:
the voltage during electrostatic spinning is 10-25 kV;
the spinning distance during the electrostatic spinning is 8-15 cm;
the advancing speed of each mixed solution during the electrostatic spinning is 6-10 mL/L; and
the receiving device during the electrostatic spinning is the metal drum with the diameter of 5 cm, and the rotation speed is 800 rpm.

18. The method of claim 15, comprising:
dissolving the drug for each nanofiber layer in a solvent, and adding polymers for each nanofiber layer in a mixture of the drug and solvent, stirring and mixing, thereby obtaining the three mixed solutions;
respectively loading the three mixed solutions into a 22G flat-head dispensing syringe for electrostatic spinning at 20-30° C., where an inner diameter of a spinneret is 0.4 mm; an advancing speed of each mixed solution is 4-10 mL/L, a spinning voltage is 10-25 kV, a spinning distance is 5-15 cm, a receiving device is a metal drum with a diameter of 5 cm; a rotation speed of the metal drum is 600-900 rpm, thus yielding a drug-loaded composite nanofiber membrane system; and
vacuum-drying the drug-loaded composite nanofiber membrane system at 20-30° C. for 24-72 h.

19. The method of claim 16, comprising:
dissolving the drug for each nanofiber layer in a solvent, and adding polymers for each nanofiber layer in a mixture of the drug and solvent, stirring and mixing, thereby obtaining the three mixed solutions;
respectively loading the three mixed solutions into a 22G flat-head dispensing syringe for electrostatic spinning at 20-30° C., where an inner diameter of a spinneret is 0.4 mm; an advancing speed of each mixed solution is 4-10 mL/L, a spinning voltage is 10-25 kV, a spinning distance is 5-15 cm, a receiving device is a metal drum with a diameter of 5 cm; a rotation speed of the metal drum is 600-900 rpm, thus yielding a drug-loaded composite nanofiber membrane system; and
vacuum-drying the drug-loaded composite nanofiber membrane system at 20-30° C. for 24-72 h.

20. A method for preparing an antitumor drug, the method comprising applying the drug-loaded composite nanofiber membrane system of claim 1.
